(19) United States
(12) Patent Application Publication
(10) Pub. No.: US 2006/0275369 A1
(43) Pub. Date: Dec. 7, 2006

(54) Orally Therapeutic Plastics and Devices Formed Therefrom

(75) Inventor: Steven D. Jensen, South Jordan, UT (US)

Correspondence Address: GEOFFREY E. DOBBIN, PATENT ATTORNEY, 4278 SOUTH 6220 WEST, WEST VALLEY CITY, UT, 84128-6501, US

(73) Assignee: CAO Group, Inc., Sandy, UT (US)

(21) Appl. No.: 11/381,320

(22) Filed: May 2, 2006

Related U.S. Application Data
(60) Provisional application No. 60/686,336, filed on Jun. 1, 2005.

(51) Int. Cl. A61K 9/14; C09D 5/16

(52) U.S. Cl. 424/486; 523/122; 264/109

(57) ABSTRACT

The present invention provides methods whereby a thermally formable plastic is compounded with releasable active ingredients. The compounded plastic is then formed into a desired dental delivery device such as an anatomical membrane, tray or strip. The dental device is then placed in the oral environment where it will release or leach active ingredients. The dental device is intended to treat, alter, improve, or aid in various conditions that are often present in the oral environment.

### CROSS REFERENCES TO RELATED APPLICATIONS

This application claims priority as a non-provisional perfection of prior filed provisional application 60/686,336 filed Jun. 1, 2005.

### FIELD OF THE INVENTION

The present invention relates to the field of therapeutic devices and more particularly relates to therapeutic devices, especially dental devices, formed of plastic that has been impregnated with beneficial active ingredients for disbursement into the oral cavity when the device is used.

### BACKGROUND OF THE INVENTION

Many dental devices have been designed and conceived in order to apply therapeutic agents to the oral cavity. Most dental devices suffer from this one drawback, they must hold a therapeutic agent, often a liquid or gel, against the teeth of a patient. One of the most common is a dental tray, which is made to conform to the shape of a mouth and is designed to hold a therapeutic agent against teeth. One of the most current devices is the use of strips either spread or coated with therapeutic gel layers. Dental trays and other devices have been designed with the concept of holding a separate agent in one locality in the mouth and have developed various ingenious methods and features to accomplish this feat. The prior art presents a serious drawback as dental devices are being manufactured which are becoming endlessly complex due to the need of controlling a separate substance. What is needed then, is a dental device where the therapeutic agent is in the device itself

### BRIEF SUMMARY OF THE INVENTION

Present invention provides methods whereby a thermally formable plastic is compounded with releasable active ingredients, including therapeutic agents. The compounded plastic is then formed into a desired therapeutic device, including dental delivery devices such as an anatomical form, tray, strip or toy. The dental device is then placed in the oral environment, of either a human or animal, where it will release or leach active ingredients.

The more important features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### DETAILED DESCRIPTION OF THE INVENTION

With reference now to the drawings, the preferred embodiment of the dental devices is herein described. It should be noted that the articles “a”, “an” and “the”, as used in this specification, include plural referents unless the content clearly dictates otherwise. The present invention in its simplest form comprises a thermally formable plastic that has been impregnated with active ingredients beneficial for the oral environment. Preferable plastics include thermoset plastics and thermoplastics or any polymer capable of being molded or formed by heat and/or pressure into a desired shape. These plastics, when cooled, will then substantially retain their desired shape.

There are many methods of forming and shaping such plastics. One economical method of molding or forming various dental devices is through injection molding. Injection molding is generally the forming or molding method of choice for thermoplastics. Injection molding generally requires the manufacture of a hollow cavity mold of any desired device. The hollow cavity is then filled under pressure with molten plastic and allowed to cool, followed by removal of the formed device from the mold.

Another economical method of molding or forming various dental devices is through compression molding. Compression molding is generally the forming or molding method of choice for thermoset plastics. Compression molding generally requires the manufacture of a mold. The polymer is introduced into the mold, followed with heat and mechanical pressure the polymer is compressed and cooled into a desired shape. Other methods of forming or molding various dental devices could also include thermoforming molding, blow molding, extrusion molding, transfer molding, reaction injection molding, or any like molding or forming method.

In general, any polymer that is capable of being formed or shaped into a dental device by the use of heat and/or pressure is within the scope of this method. A suitable polymer could be one with a low melting point in order to shield the active ingredients from destructive heat while being formed into a device. A suitable polymer should also be one that is generally deemed non-toxic or biocompatible and one that readily incorporates active ingredients internally during the compounding process, yet is able to release them in the moist oral environment. A suitable polymer should also be one that readily incorporates a non-releasable plasticizer or any substance that modifies polymeric properties.

These thermally formable plastics can also be comprised of various types of orally soluble or insoluble polymers. A dental device could be designed to be insoluble in the oral environment, yet still release active ingredients that are internally imbedded. Alternatively, a dental device could be designed such that the polymer and some or all added ingredients would dissolve in the oral environment. Such orally dissolvable devices would release imbedded active ingredients as the plastic is dissolved away by the flow of saliva. Various polymers whether soluble, insoluble, semi-soluble or combinations of these may be used to create a unique dental device with specific active ingredient releasing capabilities. Many plastics and plastic combinations are suitable for this invention. A few examples of possible plastics include: polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters, urea-formaldehyde, or any like plastics.

The characteristics or properties of these thermally formable plastics may be optionally modified by the use of plasticizers or any durometer adjusting substance. A plasticizer may be added to the plastic to adjust the final properties and characteristics favorably, such as to make the plastic softer or more pliable. A plasticizer may also be used to facilitate the metered release of active ingredients. A plasticizer may be chosen such that it will readily dissolve in saliva along with the active ingredients. A plasticizer of this type would thus aid in the release of active ingredients. Therefore, the oral release could be adjusted or metered at different rates by adding different quantities or types of plasticizers to the plastic. Many plasticizers would alter the polymeric properties to a desired plasticity and/or aid in the release of active ingredients. A few examples of possible plasticizers includes: mineral oil, triethyl citrate, acetyltriethyl citrate, lauric acid, modified vegetable oils, diacetylated monoglycerides, castor oil, sucrose diacetate hexaisobutyrate, triacetin, glycerin, liquid polyethylene glycols, liquid poly propylene glycols, propylene glycol, dimethyl phthalate, diethyl phthalate, dipropyl phthlate, dibutyl phthalate, dioctyl phthalate, polysorbates or any like or useful plasticizer.

The present invention involves the use of active ingredients that are intended to treat, alter or aid various conditions that often present themselves in the oral environment. Active ingredients may be selected to treat an oral calamity. Active ingredients may be selected to deliver a medication or drug as a preventative measure. Active ingredients may be chosen to facilitate a cosmetic procedure. Examples of more specific active ingredients may be a releasable fluoride source that is intended to treat caries disease. An active ingredient may be releasable peroxides that are intended to cosmetically whiten the teeth. An active ingredient may be an antimicrobial agent that is intended to treat an oral infection like periodontal disease. An active ingredient may be vitamins or minerals that are intended to add nutrients to a patient's diet. An active ingredient may be a sweetener and flavors that are intended to be a slow release confection.

Many active ingredients are capable of treating, altering, improving, or aiding various conditions of the oral environment. A few examples of possible active ingredients include: sodium fluoride, potassium fluoride, stannous fluoride, sodium monofluorophosphate, sucrose, fructose, glucose, corn sweeteners, aspartame, sodium saccharin, hydrogen peroxide, carbamide peroxide, chlorhexidine gluconate, thymol, propyl paraben, methyl paraben, methyl salicylate, peppermint oil, spearmint oil, chlorhexidine, sodium chlorite, vitamin E, vitamin A, ascorbic acid, soluble iron, soluble magnesium, sodium bicarbonate, sodium carbonate, sodium perborate, soluble calcium, benzalkonium chloride, xylitol, astringent alums, or any useful active ingredient for the oral environment.

These plastics are easily made in a number of methods, however the easiest is initially to add the active ingredients into the plasticizer prior to compounding the mix into plastic. Active ingredients that are soluble in a desired plasticizer are especially valuable because they become uniformly dispersed throughout the plastic during the compounding procedure along with the plasticizer. After the ingredients and plasticizer are combined, they are added to molten plastic and compounded. The resulting mass of plasticizer, active ingredients, and plastic is then mixed until substantially homogenous.

Active ingredients that are not soluble in a desired plasticizer may added directly to the molten plastic compounded by this method. Active ingredients may also be compounded into the plastic without the use of plasticizers simply by blending or mixing the molten plastic with the active ingredients.

The present invention also includes a therapeutic device that is designed to facilitate the application or delivery of the desired active ingredient. As an example, a dental device according to the present invention may be in the form of a dental tray that covers all or part of a dental arch or a thin rectangular strip that can be placed or wrapped directly over tooth surfaces. A dental device could also be a sponge that a patient activates while chewing or a thin circular disc that can be placed directly on soft tissue for targeted treatment. One particular dental device may take the shape of a thermally customizable anatomical form held in a non-thermally adjustable pre-formed holder, thereby creating a customized fit for targeted application of the ingredients. A dental device may also take the form of a small toy that would aid children or animals, such as a dog, in maintaining a treatment regimen. Any form or molded shape of a dental device that facilitates the use or application of the active ingredients is within the scope of this invention. Likewise, any form which allows contact with a body part for the therapeutic release of therapeutic agents into the body would also be included in the scope of this invention.

Upon the use or practice of this invention, it will become evident that numerous combinations and compositions are possible to produce a multitude of useful devices. A few examples of possible compositions, which would be thermally moldable and contain therapeutic agents, would include but not be limited to:

Example Formula #1

50%—polycaprolactone

0.3%—sodium saccharin

0.2%—peppermint oil

10%—aqueous hydrogen peroxide 50%

39.5%—glycerin

Example Formula #2

40%—polycaprolactone

0.3%—sodium saccharin

0.2%—peppermint oil

10%—aqueous hydrogen peroxide 50%

49.5%—triacetin

Example Formula #3

60%—polycaprolactone

0.3%—sodium saccharin

0.2%—methyl salicylate

10%—aqueous hydrogen peroxide 50%

29.5%—polysorbate 80

Example Formula #4

40%—polycaprolactone

15%—xylitol

0.2%—spearmint oil

6%—aqueous hydrogen peroxide 50%

38.8%—triethyl citrate

Example Formula #5

40%—polycaprolactone

0.3%—sodium saccharin

0.2%—peppermint oil

0.5%—sodium fluoride

59%—mineral oil

Example Formula #6

50%—poly(vinyl alcohol-co-ethylene)

25%—xylitol

0.2%—peppermint oil

24.8%—lauric acid

Example Formula #7

45%—copovidone

0.3%—sodium saccharin

0.2%—peppermint oil

8%—aqueous chlorhexidine gluconate 20%

46.5%—triacetin

Example Formula #8

40%—copovidone

0.3%—sodium saccharin

0.2%—peppermint oil

8%—aqueous hydrogen peroxide 50%

51.5%—triacetin

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method of compounding an orally therapeutic plastic:
a. An intitial step of combining at least one active ingredient with at least one plasticizer, forming a premix; and
b. A second step of combining the premix with molten plastic until homogenous.
Wherein the active ingredient will release from the plastic when placed in an oral environment.

2. The method of claim 1 the second step further comprising the addition of at least one other active ingredient in the combining process.

3. The method of claim 2, the at least one active ingredient being selected from the group of active ingredients consisting of: sodium fluoride, potassium fluoride, stannous fluoride, sodium monofluorophosphate, triclosan, sucrose, fructose, glucose, corn sweeteners, aspartame, sodium saccharin, hydrogen peroxide, carbamide peroxide, chlorhexidine gluconate, thymol, propyl paraben, methyl paraben, methyl salicylate, peppermint oil, spearmint oil, chlorhexidine, sodium chlorite, vitamin E, vitamin A, ascorbic acid, soluble iron, soluble magnesium, sodium bicarbonate, sodium carbonate, sodium perborate, soluble calcium, benzalkonium chloride, xylitol, and astringent alums.

4. The method of claim 2, the plastic being selected from the group of plastics consisting of: polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters, and urea-formaldehyde.

5. The method of claim 2, the at least one plasticizer being selected from the group of plasticizers consisting of: mineral oil, triethyl citrate, acetyltriethyl citrate, lauric acid, modified vegetable oils, diacetylated monoglycerides, castor oil, sucrose diacetate hexaisobutyrate, triacetin, glycerin, liquid polyethylene glycols, liquid poly propylene glycols, propylene glycol, dimethyl phthalate, diethyl phthalate, dipropyl phthlate, dibutyl phthalate, dioctyl phthalate, and polysorbates.

6. The method of claim 2 the second step further comprising the addition of at least one other plasticizer in the combining process.

7. The method of claim 6, the at least one active ingredient being selected from the group of active ingredients consisting of: sodium fluoride, potassium fluoride, stannous fluoride, sodium monofluorophosphate, triclosan, sucrose, fructose, glucose, corn sweeteners, aspartame, sodium saccharin, hydrogen peroxide, carbamide peroxide, chlorhexidine gluconate, thymol, propyl paraben, methyl paraben, methyl salicylate, peppermint oil, spearmint oil, chlorhexidine, sodium chlorite, vitamin E, vitamin A, ascorbic acid, soluble iron, soluble magnesium, sodium bicarbonate, sodium carbonate, sodium perborate, soluble calcium, benzalkonium chloride, xylitol, and astringent alums.

8. The method of claim 6, the plastic being selected from the group of plastics consisting of: polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters, and urea-formaldehyde.

9. The method of claim 6, the at least one plasticizer being selected from the group of plasticizers consisting of: mineral oil, triethyl citrate, acetyltriethyl citrate, lauric acid, modified vegetable oils, diacetylated monoglycerides, castor oil, sucrose diacetate hexaisobutyrate, triacetin, glycerin, liquid polyethylene glycols, liquid poly propylene glycols, propylene glycol, dimethyl phthalate, diethyl phthalate, dipropyl phthlate, dibutyl phthalate, dioctyl phthalate, and polysorbates.

10. The method of claim 1, the at least one active ingredient being selected from the group of active ingredients consisting of: sodium fluoride, potassium fluoride, stannous fluoride, sodium monofluorophosphate, triclosan, sucrose, fructose, glucose, corn sweeteners, aspartame, sodium saccharin, hydrogen peroxide, carbamide peroxide, chlorhexidine gluconate, thymol, propyl paraben, methyl paraben, methyl salicylate, peppermint oil, spearmint oil, chlorhexidine, sodium chlorite, vitamin E, vitamin A, ascorbic acid, soluble iron, soluble magnesium, sodium bicarbonate, sodium carbonate, sodium perborate, soluble calcium, benzalkonium chloride, xylitol, and astringent alums.

11. The method of claim 1, the plastic being selected from the group of plastics consisting of: polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters, and urea-formaldehyde.

12. The method of claim 1, the at least one plasticizer being selected from the group of plasticizers consisting of: mineral oil, triethyl citrate, acetyltriethyl citrate, lauric acid, modified vegetable oils, diacetylated monoglycerides, castor oil, sucrose diacetate hexaisobutyrate, triacetin, glycerin, liquid polyethylene glycols, liquid poly propylene glycols, propylene glycol, dimethyl phthalate, diethyl phthalate, dipropyl phthlate, dibutyl phthalate, dioctyl phthalate, and polysorbates.

13. A method of compounding a therapeutic plastic comprising:
a. A step of preparing a plastic for combination with other agents;
b. A step of adding at least one active ingredient to the plastic after said plastic is prepared; and
c. A step of agitating the plastic and at least one ingredient until they form a substantially homogenous blend.

14. The method of claim 13, the at least one active ingredient being selected from the group of active ingredients consisting of: sodium fluoride, potassium fluoride, stannous fluoride, sodium monofluorophosphate, triclosan, sucrose, fructose, glucose, corn sweeteners, aspartame, sodium saccharin, hydrogen peroxide, carbamide peroxide, chlorhexidine gluconate, thymol, propyl paraben, methyl paraben, methyl salicylate, peppermint oil, spearmint oil, chlorhexidine, sodium chlorite, vitamin E, vitamin A, ascorbic acid, soluble iron, soluble magnesium, sodium bicarbonate, sodium carbonate, sodium perborate, soluble calcium, benzalkonium chloride, xylitol, and astringent alums.

15. The method of claim 13, the plastic being selected from the group of plastics consisting of: polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters, and urea-formaldehyde.

16. The method of claim 13, further comprising the step of adding at least one plasticizer for combination with the plastic and at least one active ingredient.

17. The method of claim 16, the at least one active ingredient being selected from the group of active ingredients consisting of: sodium fluoride, potassium fluoride, stannous fluoride, sodium monofluorophosphate, triclosan, sucrose, fructose, glucose, corn sweeteners, aspartame, sodium saccharin, hydrogen peroxide, carbamide peroxide, chlorhexidine gluconate, thymol, propyl paraben, methyl paraben, methyl salicylate, peppermint oil, spearmint oil, chlorhexidine, sodium chlorite, vitamin E, vitamin A, ascorbic acid, soluble iron, soluble magnesium, sodium bicarbonate, sodium carbonate, sodium perborate, soluble calcium, benzalkonium chloride, xylitol, and astringent alums.

18. The method of claim 16, the plastic being selected from the group of plastics consisting of: polyacrylates, polyamide-imide, phenolic, nylon, nitrile resins, petroleum resins, fluoropolymers, copolyvidones (copovidones), epoxy, melamine-formaldehyde, diallyl phthalate, acetal, coumarone-indene, acrylics, acrylonitrile-butadiene-styrene, alkyds, cellulosics, polybutylene, polycarbonate, polycaprolactones, polyethylene, polyimides, polyphenylene oxide, polypropylene, polystyrene, polyurethanes, polyvinyl acetates, polyvinyl chloride, poly(vinyl alcohol-co ethylene), styrene acrylonitrile, sulfone polymers, saturated or unsaturated polyesters, and urea-formaldehyde.

19. The method of claim 16, the at least one plasticizer being selected from the group of plasticizers consisting of: mineral oil, triethyl citrate, acetyltriethyl citrate, lauric acid, modified vegetable oils, diacetylated monoglycerides, castor oil, sucrose diacetate hexaisobutyrate, triacetin, glycerin, liquid polyethylene glycols, liquid poly propylene glycols, propylene glycol, dimethyl phthalate, diethyl phthalate, dipropyl phthlate, dibutyl phthalate, dioctyl phthalate, and polysorbates.

20. A plastic compounded according to the method as described in any one of the preceding claims.

21. A dental device formed from the plastic of claim 20.

22. The dental device of claim 21, being a device selected from the set of devices consisting of: a dental tray, a dental form, a dental strip, an oral sponge and a dental toy.

23. A therapeutic device formed from the plastic of claim 20.
